(19) United States
(12) Patent Application Publication
(10) Pub. No.: US 2021/0310050 A1
(43) Pub. Date: Oct. 7, 2021

(54) IDENTIFICATION OF GLOBAL SEQUENCE FEATURES IN WHOLE GENOME SEQUENCE DATA FROM CIRCULATING NUCLEIC ACID

(71) Applicant: Roche Sequencing Solutions, Inc., Pleasanton, CA (US)

(72) Inventors: Mingyang Cai, Livermore, CA (US); Fergal Casey, Pleasanton, CA (US); Liang Feng, Pleasanton, CA (US); Alexander Lovejoy, Newark, CA (US)

(21) Appl. No.: 17/352,087

(22) Filed: Jun. 18, 2021

Related U.S. Application Data
(63) Continuation of application No. PCT/EP2019/086156, filed on Dec. 19, 2019.
(60) Provisional application No. 62/783,801, filed on Dec. 21, 2018.

(51) Int. Cl. C12Q 1/6806; G16B 30/10; G16B 20/20

(52) U.S. Cl. CPC C12Q 1/6806; G16B 20/20; G16B 30/10

(57) ABSTRACT

Techniques for identification of global cancer-specific sequence features in whole genome sequence data obtained from cell-free DNA (cfDNA) samples. An exemplary technique includes obtaining a plurality of whole genome sequencing reads from a cfDNA sample and determining two or more metrics from at least a majority of the plurality of genome sequencing reads, where a first metric of the two or more metrics is: (i) a fragment size of the cell free DNA, (ii) relative read depth of the plurality of whole genome sequencing reads, or (iii) germline allelic imbalance. The technique further includes inputting the two or more metrics into a classifier to obtain a first prediction for a first class and a second prediction for a second class, and classifying the sample of cell free DNA as the first class or the second class based on the first prediction and the second prediction.

### FIELD OF THE INVENTION

The present disclosure relates generally to cancer screening, and more particularly, to techniques for identification of global cancer-specific sequence features in whole genome sequence data obtained from cell-free DNA (cfDNA) samples.

### BACKGROUND

The development of plasma genotyping assays and other liquid biopsy assays has expanded the clinical utility of cell-free DNA (cfDNA) as a noninvasive cancer biomarker for cancer patient management. For example, plasma genotyping assays can noninvasively detect and quantify clinically relevant point mutations, insertions/deletions, amplifications, rearrangements, and aneuploidy within circulating tumor DNA (ctDNA) in a high background of wild-type DNA shed by nonmalignant cells. Compared to the traditional physical and biochemical methods, blood-based ctDNA detection offers a non-invasive and easily accessible way for monitoring disease state, prognostic determination, and guidance for treatment. However, as plasma genotyping assays and other liquid biopsy assays have proven utility in non-invasive ctDNA mutation detection and monitoring of minimal residual disease (MRD), there is interest in stretching the technology to determine if it has the power to discriminate the presence of cancer before a clinical diagnosis is made (i.e., cancer screening).

Currently next generation sequencing (NGS) assays for cfDNA are designed to extract information from a small targeted panel (usually <300 kb in size) that covers known oncogenes and recurrently cancer mutated positions, and such panels have had success in monitoring disease state. In some approaches, ctDNA mutations have been integrated with multiple other blood-based analytes (such as exosomes, circulating tumor cells, proteins, and metabolites) as well as integration of these signals over time for each individual in order to expand NGS assays for cfDNA to detect early stage cancer. However, for a screening application (detection of cancer prior to symptoms or before a clinical diagnosis), in contrast to monitoring, the presence or absence of specific cancer mutations is less important than finding general global cancer-specific sequence features from ctDNA sequence data that are differential between cancer and normal samples. Accordingly, new techniques are desired for cancer screening.

### BRIEF SUMMARY

Techniques are provided (e.g., a method, a system, non-transitory computer-readable medium storing code or instructions executable by one or more processors) for identification of global cancer-specific sequence features in whole genome sequence data obtained from cell-free DNA (ctDNA) samples.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect is directed to a method that includes: (a) obtaining, by a data processing system, whole genome sequence data from a sample of cell free DNA from a subject, where the whole genome sequence data includes a plurality of whole genome sequence reads. The method also includes (b) calculating, by the data processing system, two or more metrics from at least a majority of the plurality of genome sequence reads, where a first metric of the two or more metrics is (i) a fragment size of the cell free DNA, (ii) relative read depth of the plurality of whole genome sequence reads, or (iii) germline allelic imbalance. The method also includes (c) inputting, by the data processing system, the two or more metrics into a classifier to obtain a first prediction for a first class and a second prediction for a second class, where the first class is the sample of cell free DNA includes circulating tumor DNA and the second class is the sample of cell free DNA does not include the circulating tumor DNA. The method also includes (d) classifying, by the data processing system, the sample of cell free DNA as the first class or the second class based on the first prediction and the second prediction. Other embodiments of this aspect include corresponding computer systems, apparatus, and computer programs recorded on one or more computer storage devices, each configured to perform the actions of the methods.

Implementations may include one or more of the following features. The method where the second metric of the two or more metrics is: (i) a fragment size of the cell free DNA, (ii) relative read depth of the plurality of whole genome sequence reads, or (iii) germline allelic imbalance; and where the second metric is different from the first metric. The method where the classifier is a linear discriminant analysis. The method where a third metric is calculated and input into the classifier, the third metric is: (i) a fragment size of the cell free DNA, (ii) relative read depth of the plurality of whole genome sequence reads, or (iii) germline allelic imbalance; and where each of the first metric, the second metric, and the third metric are different metrics.

Implementations may include one or more of the following features. The method where the fragment size of the cell free DNA is calculated by normalizing cell free DNA fragment sizes obtained in the sample thereby obtaining a probability density function value. The method where the fragment size of the cell free DNA comprises a ratio of regions within a probability density function. The method where the ratio of regions within the probability density function comprises a ratio of probability of cell free DNA fragment size of between about 116 and about 156 nucleotides in length and a ratio of probability of cell free DNA fragment size around a mode of between about 164 and about 168 nucleotides in length.

Implementations may include one or more of the following features. The method where the fragment size of the cell free DNA is a statistical score obtained by: (i) normalizing cell free DNA fragment sizes obtained in the sample thereby obtaining a probability density function value; (ii) determining a log of values for the cell free DNA fragment sizes and first differences between consecutive cell free DNA fragment sizes; (iii) removing at least 20 of lowest cell free DNA fragment sizes to obtain a remaining cell free DNA fragment sizes; and (iv) determining a first principal component axis of the remaining cell free DNA fragment sizes as compared to cell free DNA that does include the circulating tumor DNA and a cell free DNA that does not include the circulating tumor DNA. The method where the relative read depth of the plurality of whole genome sequence reads is calculated by: (i) preprocessing of cell free DNA fragment size sequence read counts to obtain a set of normalized cell free DNA fragment size sequence read counts; (ii) determining a median read depth per chromosome arm for the set of normalized cell free DNA fragment size sequence read counts; and (iii) determining a maximum of the median read depth per chromosome arm to obtain a copy number amplification score.

Implementations may include one or more of the following features. The method where the preprocessing comprises: (i) mapping sequence read counts from various samples into windows having predetermined sizes; (ii) filtering sequence read counts in each window based on one or more factors to obtain a set of remaining cell free DNA fragment size sequence read counts for each window; (iii) correcting for guanine-cytosine content and mappability biases in each window; and (iv) normalizing remaining cell free DNA fragment size sequence read counts in each window against sequence data from cell free DNA samples that do include circulating tumor DNA. The method where the relative read depth of the plurality of whole genome sequence reads is calculated by: (i) mapping unique cell free DNA fragment size sequence read counts to obtain a cell free DNA fragment size read count distribution as measured in percentiles; and (ii) evaluating the cell free DNA fragment size read count distribution at the 99^{th} percentile or above, to determine the relative read depth of the plurality of whole genome sequence reads and obtain a copy number amplification score. The method where the relative read depth of the plurality of whole genome sequence reads is calculated by: (i) mapping unique cell free DNA fragment size sequence read counts to obtain a cell free DNA fragment size read count distribution as measured in percentiles; and (ii) determining a ratio of at least the 90^{th} percentile for sequence read count depth for each chromosome arm divided by a median sequence read count depth for each chromosome arm to obtain a copy number amplification score.

Implementations may include one or more of the following features. The method where the germline allelic imbalance is calculated using a statistical model to determine a median probability value for one or more germline allelic imbalance sites in the sample of cell free DNA, and to obtain an allelic imbalance score. The method where the statistical model comprises a binomial probability model. The method where if the median probability value for the one or more germline allelic imbalance sites is below a predetermined significance level, the median probability value is indicative of an allelic imbalance at the one or more germline sites in the sample of cell free DNA.

Implementations may include one or more of the following features. The method where the germline allelic imbalance comprises a loss of heterozygosity. The method where the sample of cell free DNA is obtained from a subject prior to a clinical diagnosis of cancer in the subject. The method where the sample of cell free DNA is obtained from a subject after a clinical diagnosis of cancer in the subject. The method further comprising predicting, by the data processing system, whether the subject has minimal residual disease based on the classification of the sample of cell free DNA as the first class or the second class. The method further comprising upon predicting the subject does have minimal residual disease, modifying a. treatment of the subject.

One general aspect is directed to a method that includes: (a) calculating two or more scores for features of whole genome sequence data obtained from a sample of cell free DNA from a subject, wherein the features include: (i) a fragment size of the cell free DNA, (ii) relative read depth of the plurality of whole genome sequence reads, (iii) germline allelic imbalance, (iv) softclipping rates, (v) rates of substitution types, (vi) overall predicted somatic mutation counts, (vii) rates of discordant reads, (vi) relative LINE/SINE element read depth, or combinations thereof; (b) inputting, by the data processing system, the two or more scores into a classifier to obtain a first prediction for a first class and a second prediction for a second class, wherein the first class is the sample of cell free DNA includes circulating tumor DNA and the second class is the sample of cell free DNA does not include the circulating tumor DNA; (c) classifying, by the data processing system, the sample of cell free DNA as the first class or the second class based on the first prediction and the second prediction; and (d) determining, by the data processing system, whether the subject has minimal residual disease based on the classification of the sample of cell free DNA as the first class or the second class.

implementations may include one or more of the following features. The method where when the sample of cell free DNA is classified as the first class, determining that the subject has minimal residual disease. The method where when the sample of cell free DNA is classified as the second class, determining that the subject does not have minimal residual disease. The method further includes predicting, by the data processing system, a clinical outcome of a treatment regimen for the subject based upon whether the subject has the minimal residual disease; and upon determining the subject does have minimal residual disease and predicting a negative clinical outcome, modifying the treatment regimen of the subject. Other embodiments of this aspect include corresponding computer systems, apparatus, and computer programs recorded on one or more computer storage devices, each configured to perform the actions of the methods.

The techniques described above and below may be implemented in a number of ways and in a number of contexts. Several example implementations and contexts are provided with reference to the following figures, as described below in more detail. However, the following implementations and contexts are but a few of many.

### BRIEF DESCRIPTION OF THE DRAWINGS

FIG. 1 depicts a flowchart illustrating a process for identification of global cancer-specific sequence features in whole genome sequence data obtained from cell-free DNA samples in accordance with various embodiments.

FIG. 2 depicts a block diagram of a sequence analytical system in accordance with various embodiments.

FIG. 3 depicts a block diagram of a computing system or data processing system in accordance with various embodiments.

FIG. 4 depicts a flowchart illustrating a process for calculating a fragment score in accordance with various embodiments.

FIG. 5 depicts a flowchart illustrating a process for calculating a copy number amplification score in accordance with various embodiments.

FIG. 6 depicts the effect of preprocessing to remove the noise from the coverage profiles in a cancer sample and normal sample in accordance with various embodiments.

FIGS. 7A-7C depict univariate analysis of feature summaries to separate out colon and lung cancer datasets from normal samples in accordance with various embodiments.

FIG. 8 depicts the Receiver Operator Characteristics (ROC) area under the curve (AUC) for a classifier used in a multivariate analysis of feature summaries in accordance with various embodiments.

FIG. 9 depicts an LDA score and CNA score for a number of samples from individuals that are healthy, presenting with colon cancer, or presenting with lung cancer in accordance with various embodiments.

FIG. 10 depicts a flowchart illustrating a process for diagnosing a patient with minimal residual disease in accordance with various embodiments.

### DETAILED DESCRIPTION

In the following description, various embodiments will be described. For purposes of explanation, specific configurations and details are set forth in order to provide a thorough understanding of the embodiments. However, it will also be apparent to one skilled in the art that the embodiments may be practiced without the specific details. Furthermore, well-known features may be omitted or simplified in order not to obscure the embodiment being described.

### I. Introduction

In various embodiments, techniques are provided (e.g., a method, a system, non-transitory computer-readable medium storing code or instructions executable by one or more processors) for screening for cancer using sequence data obtained from circulating nucleic acid. In some embodiments, the circulating nucleic acid is ctDNA, which originates directly from the tumor or from circulating tumor cells (CTCs), which are viable, intact tumor cells that shed from primary tumors and enter the bloodstream or lymphatic system. ctDNA is different from cfDNA, which is a broader term that describes DNA that is freely circulating in the bloodstream, but is not necessarily of tumor origin. Because ctDNA may reflect the entire tumor genome, it has gained traction for its potential clinical utility. For example, liquid biopsies of ctDNA may be obtained in a noninvasive form such as blood draws at various time points to monitor tumor progression throughout the treatment regimen.

More recently, researchers have expanded on the conventional use of liquid biopsies of ctDNA to screen for common cancer types. A used herein, a “screen” or “screening” for a disease or condition such as cancer refers to a technique for determining the likely presence or absence of the disease or condition in a subject not presenting with symptoms or not previously diagnosed with the disease or condition. These assays simultaneously evaluate levels of blood-based analytes (such as exosomes, circulating tumor cells, proteins, and metabolites) and the presence of cancer gene mutations from cfDNA in the blood. The mutation panel in the assays used for identifying the cancer gene mutations is intentionally kept small to minimize false-positive results and keep the assays affordable. A problem associated with these assays is that ctDNA in circulation is more representative in patients with advanced and metastatic than local diseases, and the likelihood of finding mutations increases alongside the aggressiveness of the disease, and thus the mutation panel in these assays is not always sensitive enough to detect cancer-related genetic alterations in the cDNA. Moreover, the multianalyte approach is critical for developing the screening assays with adequate sensitivity, as the presence of the blood-based analytes or the cancer gene mutations alone is not sufficiently sensitive to screen for cancer.

To address these problems, various embodiments disclosed herein are directed to techniques for the identification of global cancer-specific sequence features in whole genome sequence data obtained from circulating nucleic acid. The techniques combine the global cancer-specific sequence features in a multivariate classifier to predict whether a sample comprising the circulating nucleic acid includes ctDNA, and optionally, withholds a set of reference normals to systematically model sequencing background variability. The global cancer-specific sequence features are independent of particular mutations present in the circulating nucleic acid, and, as demonstrated herein, have been determined to accurately distinguish between cancer and non-cancer samples. Surprisingly it has been discovered that some of the cancer samples that are identified by these techniques do not have a single somatic mutation detected by conventional ctDNA mutation panels, and thus these techniques actually make ctDNA visible in the absence of detectable mutations.

One illustrative embodiment of the present disclosure comprises: (a) obtaining, by a data processing system, whole genome sequence data from a sample of cell free DNA from a subject, where the whole genome sequence data includes a plurality of whole genome sequence reads. The method also includes (b) determining, by the data processing system, two or more metrics from at least a majority of the plurality of genome sequence reads, where a first metric of the two or more metrics is (i) a fragment size of the cell free DNA, (ii) relative read depth of the plurality of whole genome sequence reads, or (iii) germline allelic imbalance. The method also includes (c) inputting, by the data processing system, the two or more metrics into a classifier to obtain a first prediction for a first class and a second prediction for a second class, where the first class is the sample of cell free DNA includes circulating tumor DNA and the second class is the sample of cell free DNA does not include the circulating tumor DNA. The method also includes (d) classifying, by the data processing system, the sample of cell free DNA as the first class or the second class based on the first prediction and the second prediction. As used herein, when an action is “triggered by” or “based on” something, this means the action is triggered or based at least in part on at least a part of the something.

Advantageously, these approaches expand on the conventional use of liquid biopsies of ctDNA to screen for cancer without relying on mutation detection. Moreover, these approaches have been demonstrated to not only accurately distinguish between cancer and non-cancer samples but also detect cancer in samples that do not have a single somatic mutation detected. Consequently, these approaches are capable of making ctDNA visible in the absence of detectable mutations.

### II. Techniques for the Identification of Global Cancer-Specific Sequence Features in Whole Genome Sequence Data Obtained From Circulating Nucleic Acid

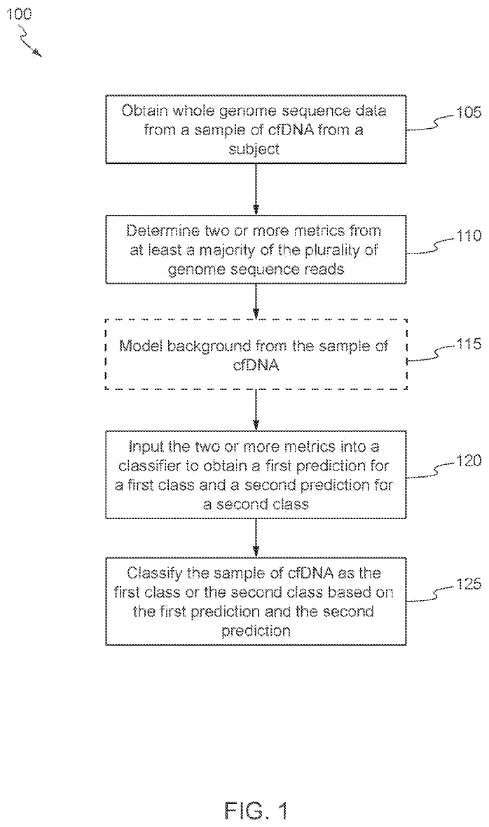

FIG. 1 illustrates processes and operations for the identification of global cancer-specific sequence features in whole genome sequence data obtained from circulating nucleic acid. Individual embodiments may be described as a process which is depicted as a flowchart, a flow diagram, a data flow diagram, a structure diagram, or a block diagram. Although a flowchart may describe the operations as a sequential process, many of the operations may be performed in parallel or concurrently. In addition, the order of the operations may be re-arranged. A process is terminated when its operations are completed, but could have additional steps not included in a figure or the description thereof. A process may correspond to a method, a function, a procedure, a subroutine, a subprogram, etc. When a process corresponds to a function, its termination may correspond to a return of the function to the calling function or the main function.

The processes and/or operations depicted in FIG. 1 may be implemented in software (e.g., code, instructions, program) executed by one or more processing units (e.g., processors cores), hardware, or combinations thereof. The software may be stored in a memory (e. g., on a memory device, on a non-transitory computer-readable storage medium). The particular series of processing steps in FIG. 1 is not intended to be limiting. Other sequences of steps may also be performed according to alternative embodiments. For example, in alternative embodiments the steps outlined herein may be performed in a different order. Moreover, the individual steps illustrated in FIG. 1 may include multiple sub-steps that may be performed in various sequences as appropriate to the individual step. Furthermore, operations or steps may be added or removed depending on the particular applications. One of ordinary skill in the art would recognize many variations, modifications, and alternatives.

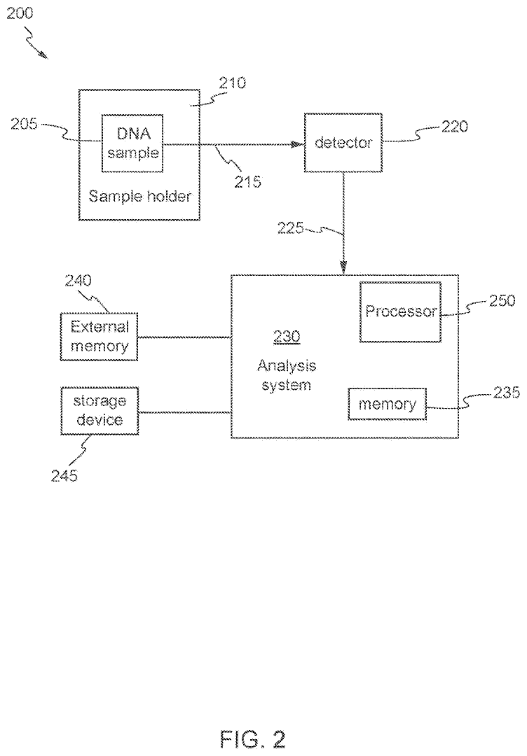

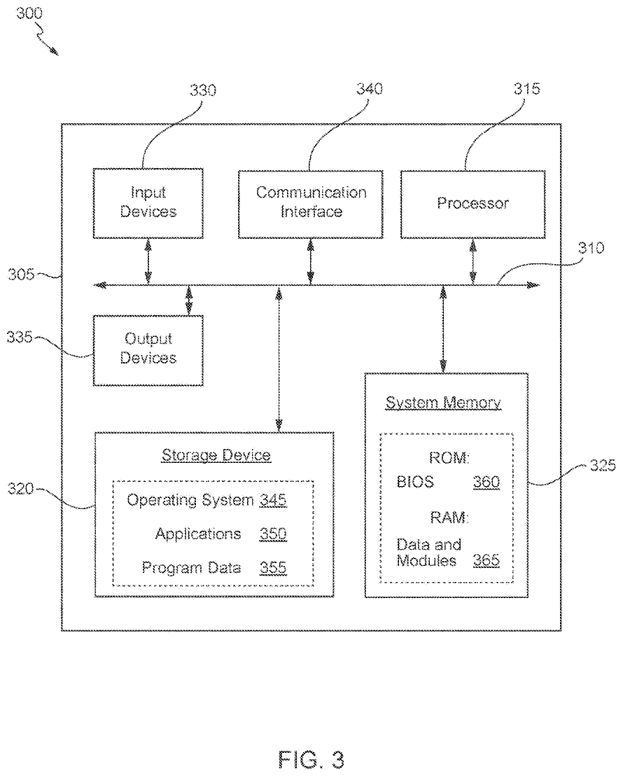

FIG. 1 shows a flowchart 100 that illustrates a process for the identification of global cancer-specific sequence features in whole genome sequence data obtained from circulating nucleic acid. In some embodiments, the processes depicted in flowchart 100 may be implemented by the architecture, systems, and techniques depicted in FIGS. 2 and 3. At step 105, whole genome sequence data is obtained from a sample of cfDNA from a subject (e.g., a patient). The whole genome sequence data includes a plurality of whole genome sequence reads. The sequence reads may be obtained by single-end or paired-end sequencing and analyzed using any suitable sequencing technique, as described in detail in Section III. In some embodiments, one or more samples having cfDNA are obtained (e.g., by drawing blood from a subject), sequenced, by a sequence analytical system, to generate sequence data for the cfDNA, and the sequence data is analyzed, by a data processing system, to provide some output such as tumor burden and a statistical significance of a tumor burden. In other embodiments, the sequence data is obtained, by the data processing system, from any source (public or private) in a suitable manner, and analyzed, by the data processing system, to provide some output such as fragment size of the cfDNA, relative read depth of the plurality of whole genome sequence reads, or germline allelic imbalance. In some embodiments, the sample of cfDNA is obtained from a subject prior to a clinical diagnosis of cancer in the subject. In other embodiments, the sample of cfDNA is obtained from the subject after a clinical diagnosis of cancer in the subject.

Whole genome sequencing (also known as WGS, full genome sequencing, complete genome sequencing, or entire genome sequencing) is the process of determining the complete DNA sequence of an organism's genome at a single time. For example, cfDNA can be taken by simple venipuncture from a subject and used for whole genome sequencing of the subject. In some embodiments, the whole genome sequencing is low-pass whole-genome sequencing to generate low coverage whole genome sequence data for the cfDNA. As used herein, “coverage” (or depth) in DNA sequencing is the number of unique reads that include a given nucleotide in the reconstructed sequence. Deep sequencing refers to the general concept of aiming for a high number of unique reads of each region of a sequence (e.g., >100×), and is typically used for mutant detection in cfDNA. In contrast, as used herein, “low-pass” sequencing refers to a genome that is sequenced to a depth under 10×.

At step 110, two or more metrics are calculated from at least a majority of the plurality of genome sequence reads. A used herein, a “majority” is the greater part, or more than half, of the total. For example, a majority is a subset of the plurality of genome sequence reads consisting of more than half of the plurality of genome sequence reads. In some embodiments, a first metric of the two or more metrics is: (i) a fragment size of the cfDNA, (ii) relative read depth of the plurality of whole genome sequence reads, or (iii) germline allelic imbalance. In some embodiments, a second metric of the two or more metrics is: (i) a fragment size of the cfDNA, (ii) relative read depth of the plurality of whole genome sequence reads, or (iii) germline allelic imbalance, and the second metric is different from the first metric. In some embodiments, a third metric of the two or more metrics is: (i) a fragment size of the cfDNA, (ii) relative read depth of the plurality of whole genome sequence reads, or (iii) germline allelic imbalance, and each of the first metric, the second metric, and the third metric are different metrics,

In various embodiments, the fragment size of the cfDNA is calculated by normalizing ctDNA fragment sizes obtained in the sample thereby obtaining a probability density function value. As used herein, the “fragment size” refers to a count or an average of the number of base pairs of the insert and the adapters that make up teach fragment. As used herein, the “insert” is the base pairs between the adapters, and the “insert size” is the count or an average of the number of base pairs of the insert. The technique of obtaining a probability density function value exploits apparent size differences between ctDNA and cfDNA. Specifically, previous studies have shown that ctDNA is highly fragmented and occurs most commonly at a size <100 bp, while normal cell-free DNA is proportionally more represented at a size >400 bp. Thus, in order to detect ctDNA associated with cancer, a probability density function value may be used to exploit differences in fragment sizes to separate ctDNA from the background ctDNA, as described in detail in Section IV. In so doing, precise differences in fragment lengths between ctDNA and cfDNA may be identified. In some embodiments, the fragment size of the cfDNA comprises a ratio of regions within a probability density function. In some embodiments, the ratio of regions within the probability density function comprises a ratio of probability of cfDNA fragment size of between about 116 and about 156 nucleotides in length and a ratio of probability of cfDNA fragment size around a mode of between about 164 and about 168 nucleotides in length. In certain embodiments, the fragment size of the cfDNA is a statistical fragment score obtained by: (i) normalizing cfDNA fragment sizes obtained in the sample thereby obtaining a probability density function value; (ii) determining a log of values for the cfDNA fragment sizes and first differences between consecutive cfDNA fragment sizes; (iii) removing at least 20 of lowest cfDNA fragment sizes to obtain a remaining cfDNA fragment sizes; and (iv) determining a first principal component axis of the remaining cfDNA fragment sizes as compared to cfDNA that does include the ctDNA and a cfDNA that does not include the ctDNA.

In various embodiments, relative read depth of the plurality of whole genome sequence reads is calculated by: (i) preprocessing of cell free DNA fragment size sequence read counts to obtain a set of normalized cell free DNA fragment size sequence read counts; (ii) determining a median read depth per chromosome arm for the set of normalized cfDNA fragment size sequence read counts; and (iii) determining a maximum of the median read depth per chromosome arm to obtain a copy number amplification score, as described in detail in Section V. In some embodiments, preprocessing of the cfDNA comprises: (i) mapping sequence read counts from various samples into windows having predetermined sizes; (ii) filtering sequence read counts in each window based on one or more factors to obtain a set of remaining cfDNA fragment size sequence read counts for each window; (iii) correcting for guanine-cytosine content and mappability biases in each window; and (iv) normalizing remaining cfDNA fragment size sequence read counts in each window against sequence data from cfDNA samples that do include ctDNA. In other embodiments, the relative read depth of the plurality of whole genome sequence reads is calculated by: (i) mapping unique ctDNA fragment size sequence read counts to obtain a cfDNA fragment size read count distribution as measured in percentiles; and (ii) evaluating the ctDNA fragment size read count distribution at the 99^{th} percentile or above, to determine the relative read depth of the plurality of whole genome sequence reads and obtain a copy number amplification score. In other embodiments, the relative read depth of the plurality of whole genome sequence reads is calculated by: (i) mapping unique cfDNA fragment size sequence read counts to obtain a cfDNA fragment size read count distribution as measured in percentiles; and (ii) evaluating the cfDNA fragment size read count distribution at the 99^{th} percentile or above, to determine the relative read depth of the plurality of whole genome sequence reads and obtain a copy number amplification score.

In various embodiments, a germline allelic imbalance is calculated using a statistical model to obtain a median probability value for one or more germline allelic imbalance sites in the sample of cfDNA, and to obtain an allelic imbalance score, as described in detail in Section VI. In some embodiments, the statistical model comprises a binomial probability model. In some embodiments, if the median probability value for the one or more germline allelic imbalance sites is below a predetermined significance level, the median probability value is indicative of an allelic imbalance at the one or more germline sites in the sample of cfDNA. In certain embodiments, the germline allelic imbalance comprises a loss of heterozygosity.

In various embodiments, the two or more metrics comprise: (i) a fragment size of the cfDNA, (ii) relative read depth of the plurality of whole genome sequence reads, (iii) germline allelic imbalance, (iv) softclipping rates, (v) rates of substitution types, (vi) overall predicted somatic mutation counts, (vii) rates of discordant reads, (vi) relative LINE/SINE element read depth, or combinations thereof. The fragment size, relative read depth, and allelic imbalance may be calculated as the fragment score, the copy number amplification score, and the allelic imbalance score, respectively (as discussed in Sections IV, V, and VI). The metrics that are rates may be calculated as percentages of reads/variants (out of total) reads that fall into the given category. In some embodiments, a first metric of the two or more metrics is the fragment score, and a second metric of the two or more metrics is the copy number amplification score, or the allelic imbalance score. In some embodiments, a first metric of the two or more metrics is the fragment score, a second metric of the two or more metrics is the copy number amplification score, and a third metric of the two or more metrics is the allelic imbalance score. In some embodiments, the determination of the two or more metrics is part of a whole genome sequencing data analysis pipeline that carries out the standard quality control steps (e.g., fastq quality check, adapter trimming, duplicate removal) and computes the two or more metrics from at least a majority of the plurality of genome sequence reads for downstream analysis.

At optional step 115, background in the sample of cfDNA is modeled. In some embodiments, the modeling comprises using prior information on a set aside normal reference set to identify genomic regions that are “clean”. That is, for example, if a global fragment size score is being defined, then a set of reference normals would be initially examined and regions of the reference normals would be identified that consistently have fragment size scores greater than a predetermined threshold such as >200 bp or >400 bp. The regions of the reference normal may then be used to filter out the background signal in subsequent processing, The same type of preselection can be used for identifying regions where discordant reads are absent or almost absent in normals. This way the background signal is kept as low as possible in normals and sensitivity and specificity are improved for classifying the sample of cfDNA. At step 120, the two or more metrics are input into a classifier to obtain a first prediction for a first class and a second prediction for a second class, as described in detail in Section VII. In some embodiments, the first class is the sample of cfDNA includes circulating tumor DNA and the second class is the sample of cfDNA does not include the circulating tumor DNA. In certain embodiments, the classifier is a linear discriminant analysis. In some embodiments, the background is filtered from the classifier based on the modeling from step 115. At step 125, the sample of cfDNA is classified as the first class or the second class based on the first prediction and the second prediction.

### Sequencing Samples and Analysis System

FIG. 2 shows an example sequence analytical system 200 used in accordance with various embodiments that includes a sample 205, such as a blood sample comprising cfDNA, within a sample holder 210, e.g., a flow cell or a tube containing droplets of cfDNA. A physical characteristic 215, such as a fluorescence intensity value, from the sample 205 is detected by a detector 220. A data signal 225 from the detector 220 can be sent to a data processing system 230 (onboard or separate from the detector), which may include a processor 250 and a memory 235. Data signal 225 may be stored locally in the data processing system 230 in memory 235, or externally in an external memory 240 or a storage device 245. Detector 220 can detect a variety of physical signals, such as light (e.g., fluorescent light from different probes for different bases) or electrical signals (e.g., as created from a molecule traveling through a nanopore). The data processing system 230 may be, or may include, a computer system, ASIC, microprocessor, etc., as described in further detail with respect to FIG. 3. The data processing system 230 may also include or be coupled with a display (e.g., monitor, LED display, etc.) and a user input device (e.g., mouse, keyboard, buttons, etc.). The data processing system 230 and the other components may be part of a stand-alone or network connected computer system, or they may be directly attached to or incorporated in a thermal cycler device. The data processing system 230 may also include optimization software that executes in processor 250. Based on the sequence data, mutations in one or more reads may be quantified and analyzed to determine a tumor burden and a statistical significance of a tumor burden.

Any of the computer systems or data processing systems described herein may utilize any suitable number of subsystems. An example of a computer system or data processing system (e.g., the data processing system 230 described with respect to FIG. 2) and associate subsystems is shown in FIG. 3. The computing system 300 is only one example of a suitable computing system and is not intended to suggest any limitation as to the scope of use or functionality of the present embodiments. Also, computing system 300 should not be interpreted as having any dependency or requirement relating to any one or combination of components illustrated in sequence analytical system 200.

As shown in FIG. 3, computing system 300 includes a computing device 305. The computing device 305 can be resident on a network infrastructure such as within a cloud environment, or may be a separate independent computing device (e.g., a computing device of a service provider). The computing device 305 may include a bus 310, processor 315, a storage device 320, a system memory (hardware device) 325, one or more input devices 330, one or more output devices 335, and a communication interface 340.

The bus 310 permits communication among the components of computing device 305. For example, bus 310 may be any of several types of bus structures including a memory bus or memory controller, a peripheral bus, and a local bus using any of a variety of bus architectures to provide one or more wired or wireless communication links or paths for transferring data and/or power to, from, or between various other components of computing device 305.

The processor 315 may be one or more conventional processors, microprocessors, or specialized dedicated processors that include processing circuitry operative to interpret and execute computer readable program instructions, such as program instructions for controlling the operation and performance of one or more of the various other components of computing device 305 for implementing the functionality, steps, and/or performance of the present invention. In certain embodiments, processor 315 interprets and executes the processes, steps, functions, and/or operations of the present invention, which may be operatively implemented by the computer readable program instructions. For example, processor 315 can retrieve, e.g., import and/or otherwise obtain or generate sequence data, query the sequence data, determine or calculate metrics, model background, determine probability values, provide predications such as classes, interpretive diagnosis, and clinical outcomes. In embodiments, the information obtained or generated by the processor 315, e.g., the sequence data, the metrics, background models, probability values, classes, etc., can be stored in the storage device 320.

The storage device 320 may include removable/non-removable, volatile/non-volatile computer readable media, such as, but not limited to, non-transitory machine readable storage medium such as magnetic and/or optical recording media and their corresponding drives. The drives and their associated computer readable media provide for storage of computer readable program instructions, data structures, program modules and other data for operation of computing device 305 in accordance with the different aspects of the present invention. In embodiments, storage device 320 may store operating system 345, application programs 350, and program data 355 in accordance with aspects of the present invention.

The system memory 325 may include one or more storage mediums, including for example, non-transitory machine readable storage medium such as flash memory, permanent memory such as read-only memory (“ROM”), semi-permanent memory such as random access memory (“RAM”), any other suitable type of non-transitory storage component, or any combination thereof. In some embodiments, an input/output system 360 (BIOS) including the basic routines that help to transfer information between the various other components of computing device 305, such as during start-up, may be stored in the ROM. Additionally, data and/or program modules 365, such as at least a portion of operating system 345, program modules, application programs 350, and/or program data 355, that are accessible to and/or presently being operated on by processor 315, may be contained in the RAM. In embodiments, the program modules 365 and/or application programs 350 can comprise an index or table of metrics, algorithms or models such as a Monte Carlo algorithm to model background, a classifier such as linear discriminant analysis, and a comparison tool, which provides the instructions for execution of processor 315.

The one or more input devices 330 may include one or more mechanisms that permit an operator to input information to computing device 305, such as, but not limited to, a touch pad, dial, click wheel, scroll wheel, touch screen, one or more buttons (e.g., a keyboard), mouse, game controller, track ball, microphone, camera, proximity sensor, light detector, motion sensors, biometric sensor, and combinations thereof. The one or more output devices 335 may include one or more mechanisms that output information to an operator, such as, but not limited to, audio speakers, headphones, audio line-outs, visual displays, antennas, infrared ports, tactile feedback, printers, or combinations thereof.

The communication interface 340 may include any transceiver-like mechanism (e.g., a network interface, a network adapter, a modem, or combinations thereof) that enables computing device 305 to communicate with remote devices or systems, such as a mobile device or other computing devices such as, for example, a server in a networked environment, e.g., cloud environment. For example, computing device 305 may be connected to remote devices or systems via one or more local area networks (LAN) and/or one or more wide area networks (WAN) using communication interface 340.

s discussed herein, computing system 300 may be configured for ultrasensitive detection of circulating nucleic acid with prior knowledge of variants to be monitored in the blood. In particular, computing device 305 may perform tasks (e.g., process, steps, methods and/or functionality) in response to processor 315 executing program instructions contained in non-transitory machine readable storage medium, such as system memory 325. The program instructions may be read into system memory 325 from another computer readable medium (e.g., non-transitory machine readable storage medium), such as data storage device 320, or from another device via the communication interface 340 or server within or outside of a cloud environment. In embodiments, an operator may interact with computing device 305 via the one or more input devices 330 and/or the one or more output devices 335 to facilitate performance of the tasks and/or realize the end results of such tasks in accordance with aspects of the present invention. In additional or alternative embodiments, hardwired circuitry may be used in place of or in combination with the program instructions to implement the tasks, e.g., steps, methods and/or functionality, consistent with the different aspects of the present invention. Thus, the steps, methods and/or functionality disclosed herein can be implemented in any combination of hardware circuitry and software.

### IV. Fragment Metrics

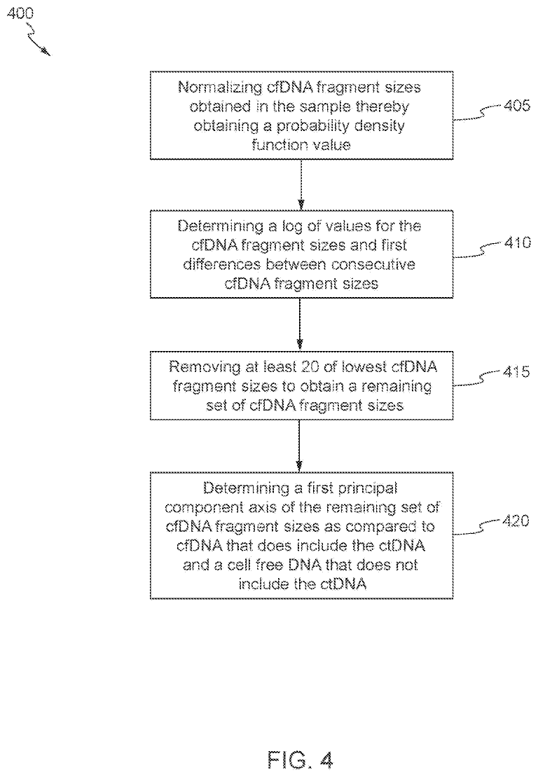

In various embodiments, a fragment score is calculated that is associated with a fragment size of the sequence reads. It has been discovered that DNA fragment size is smaller in ctDNA fragments compared to healthy cfDNA. In some embodiments, a fragment size distribution is determined to calculate the fragment score for fragment size, as shown in FIG. 4. The distribution of the fragment size for the cfDNA sample(s) is determined as a listing or function showing all the possible sizes (e.g., numbers of base pairs) of the sequence data and how often each fragment size is occurring. At step 405, the fragment size distribution for the sample is normalized such that the distribution is a probability density function (e.g., a function describing the probability that a given size will occur, and the function describing the cumulative probability that a given size or any size smaller than it will occur is a distribution function). The probability density function of the fragment size distribution may be defined as the derivative of the distribution function. In certain embodiments, the normalizing is performed with a linear transformation such that the fragment size data obtained from the sequence data is resealed to a unit interval. At step 410, a logarithmic transformation (taking a log) is performed on the fragment size values, and first differences between consecutive insert size lengths are obtained. This provides information on the shape of the fragment distribution. At step 415, the fragment sizes are filtered by removing the first 20, 30, 40, or 50 fragment length values (these counts may be too low and noisy). In certain embodiments, between 10 and 60 fragment length values are removed to filter the fragment sizes.

At step 420, a first principal component axis of the fragment length distributions is calculated across a collection of normal and cancer samples. This step may be performed as a part of a number of preprocessing steps to emphasize variation and bring out strong patterns in fragment size datasets. In certain embodiments, the fragment size is made the first principal component and the second principal component (e.g., the number of same fragment sizes) is dropped. The results of the principal component analysis provides component scores or factor scores (the transformed variable values corresponding to a particular data point), and loadings (the weight by which each standardized original variable should be multiplied to get the fragment score). In certain embodiments, the first principal component provides the weighting (loadings) to give insert size value in the test data (see, e.g., the cfDNA sample being processed step 105).

In other embodiments, alternative summarizations are possible for fragment distributions to enhance detection of cancer samples. Fax example, a lower quantile (0.1%, 1%, 5%) of distribution may be used to calculate the fragment score for the fragment size. Alternatively, a probability density function value at a set cutoff or within a range may be used to calculate the fragment score for the fragment size, e.g., a probability density function at fragment length of 120 bp, 130 bp, or 140 bp, or a fixed number of units less than the mode of the distribution (e.g., usually the mode of distribution is about 166 bp for cfDNA), sum of probability density function values between 50 units and 10 units less than the mode (e.g., between about 116 bp to about 156 bp). Alternatively, ratio of regions within the probability density function may be used to calculate the fragment score for the fragment size, e.g., a ratio of probability of fragment length lying between about 116 bp and about 156 bp to the probability of fragment length around the mode between about 164 bp and about 168 bp, as relative enrichment may be expected of the lower fragment lengths. As used herein, the term “substantially,” “approximately,” or “about” may be substituted with “within [a percentage] of ” what is specified, where the percentage includes 0.1, 1, 5, and 10 percent.

### V. Relative Read Depth Metrics

In various embodiments, a copy number amplification score is calculated that is associated with a measure of copy number variation in the sequence reads. It has been discovered that the presence of copy number amplification (increased copy number alterations or variation) is found more frequently in ctDNA fragments as compared to healthy cfDNA. Relative read depth is intended to assess the presence of focal or broad copy number changes within a ctDNA sample. Therefore, as used herein, “relative read depth” is a measure of copy number variation. In some embodiments, the relative read depth of a plurality of whole genome sequence reads is calculated, as shown in FIG. 5. In some embodiments, the calculation comprises step 505 where a number of preprocessing steps are performed to remove the noise from the coverage profiles and obtain a set of normalized cfDNA fragment size sequence read counts. At step 505a, sequence read counts from various cfDNA samples (e.g., cfDNA that does include ctDNA and a cDNA that does not include ctDNA) are mapped into bins or windows having predetermined sizes. The sequence read counts are the number of reads for each probe used in the sequencing, and may optionally be corrected according to a one or more different known factors to correct for any bias. In certain embodiments, the bin or window sizes are between 10 kb and 10000 kb, for example 200 kb. At step 505b, sequence read counts are filtered in each window based on one or more factors to obtain a set of remaining cfDNA fragment size sequence read counts for each window. The filtering includes dropping the sequence read counts from subsequent analysis. In some embodiments, the one or more factors include sequence counts that are less than a predetermined threshold. In certain embodiments, the predetermined threshold is less than 350 sequence reads, for example, less than 200 sequence reads. In some embodiments, the one or more factors include centromeric reads. In some embodiments, the one or more factors include sequence reads having a variable cytoband.

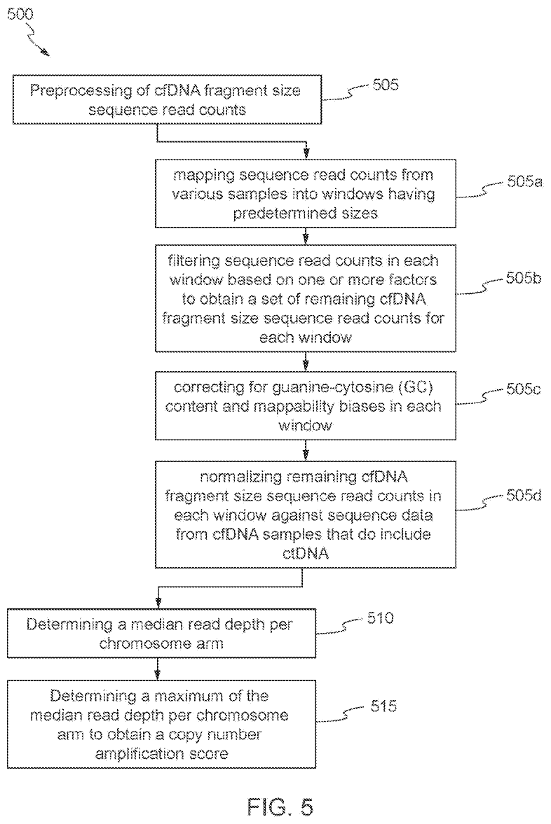

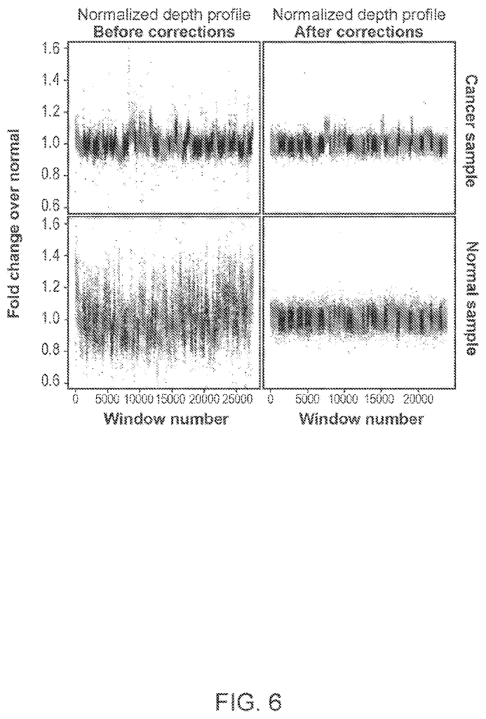

At step 505c, guanine-cytosine (GC) content and mappability biases are corrected in each window. The GC content bias describes the dependence between fragment count (read coverage) and GC content found in sequencing data. The GC bias can dominate the signal of interest for analyses that focus on measuring fragment abundance within a genome if nor corrected. The read mapping procedure generates regional mappability bias. Because sequence reads that map to multiple sites in the genome are usually discarded, genomic regions with high sequence degeneracy show lower mapped read coverage than unique regions, creating systematic bias if not corrected. At step 505d, remaining cfDNA fragment size sequence read counts in each window are normalized against sequence data from cfDNA samples that do include ctDNA. The resulting cleanup is shown in a cancer and normal sample in FIG. 6.

After preprocessing is completed a number of genomewide summaries can be evaluated on the normalized depth data (i.e., the set of normalized cfDNA fragment size sequence read counts). In some embodiments, a summarization of the normalized depth data is a maximum of the median normalized depths by chromosome arm. For example, the relative read depth calculation may further comprise step 510 where a median read depth per chromosome arm is determined for the set of normalized cfDNA fragment size sequence read counts, and step 515 where a maximum of the median read depth per chromosome arm is determined to obtain a copy number amplification score, which captures arm level amplifications. In other embodiments, the summarization of the normalized depth data is the high percentiles of the binned or windowed values such as the 99th percentile, 99.9th percentile and 99.99th percentile. The relative read depth of the plurality of whole genome sequence reads may be calculated by: (i) mapping unique cfDNA fragment size sequence read counts to obtain a cfDNA fragment size read count distribution as measured in percentiles; and (ii) evaluating the cell free DNA fragment size read count distribution at the 99^{th} percentile or above, to determine the relative read depth of the plurality of whole genome sequence reads and obtain a copy number amplification score. In other embodiments, the summarization of the normalized depth data is the ratios of high percentiles divided by median depth per chromosome arm in order to identify focal amplifications. For example, the 90th percentile for depth for each chromosome arm divided by the median depth for each arm. The relative read depth of the plurality of whole genome sequence reads is calculated by: (i) mapping unique cfDNA fragment size sequence read counts to obtain a cfDNA fragment size read count distribution as measured in percentiles; and (ii) determining a ratio of at least the 90^{th} percentile for sequence read count depth for each chromosome arm divided by a median sequence read count depth for each chromosome arm to obtain a copy number amplification score.

### VI. Germline Allelic Imbalance Metric

In various embodiments, an allelic imbalance score is calculated that is associated with a measure of copy number variation in the sequence reads. It has been discovered that the presence of germline imbalances is found more frequently in ctDNA fragments as compared to healthy cfDNA. For heterozygous single nucleotide polymorphisms (SNPs) in a region of normal copy number the expected allele frequency (AF) is 50%. For regions with copy number gain or loss that AF may be distorted away from 50%, e.g., with 3 copies at a locus the heterozygous SNPs will be at ⅔=66% or ⅓=33% AF. This is referred to as “germline allelic imbalance,” and a formula for calculating a score for the allelic imbalance is provided based on a binomial probability. In some embodiments, the allelic imbalance score is calculated using a statistical model to obtain a median probability value for one or more germline allelic imbalance sites in the sample of cell free DNA. In some embodiments, the statistical model comprises: a null hypothesis where the germline variant is heterozygous and the probability of seeing the germline variant in a given read at a site is a significance level of p₀=0.5, then for an observation of y_{obs} non-reference reads out of n reads total at a site, the p-value for rejecting the null hypothesis is given by the following equations (1-3):

$$\begin{matrix}{π}_{obs} = P ( Y = {y}_{obs} \vert p = {p}_{0} ) = ( \begin{matrix}n \\ {y}_{obs}\end{matrix} ) {{p}_{0}^{{y}_{obs}} ( 1 - {p}_{0} )}^{n - {y}_{obs}} & ( Equation 1 ) \\ {π}_{j} = P ( Y = j \vert p = {p}_{0} ) = ( \begin{matrix}n \\ j\end{matrix} ) {{p}_{0}^{j} ( 1 - {p}_{0} )}^{n - j} , j = 0 , … , n & ( Equation 2 ) \\ p - value = {\sum }_{j = 0}^{n} {π}_{j} I ( {π}_{j} \leq {π}_{obs} ) , \\ where ( {π}_{j} \leq {π}_{o b s} ) = \{ \begin{matrix}1 & if {π}_{j} \leq {π}_{o b s} \\ 0 & if {π}_{j} > {π}_{o b s}\end{matrix} & ( Equation 3 )\end{matrix}$$

In this statistical model, the probability values (p-values) are created per germline site and used as the allelic imbalance score. For example, a low median p-value across the entire sample is indicative of germline imbalances. Alternatively, since germline allelic imbalance should relate to copy number changes, the allelic imbalance score may be defined as the correlation of the allelic imbalances with the normalized depths, and a correlation would arise as low germline allelic imbalance p-values would correspond to either high (amplification) or low (heterozygous deletion) normalized depths.

### VII. Univariate and Multivariate Experiments and Analysis

Proof-of-concept experiments and analysis were performed for low coverage (average depths ranging from 1 to 5) whole genome sequence (WGS) data of cfDNA from cancer samples (25 stage IV lung cancer, and 25 metastatic CRC samples) as well as a cfDNA from healthy controls (24 samples) to evaluate potential global sequence read features that are indicative of presence of cancer derived cfDNA. Although these are late stage cancers, inferred ctDNA content from a matched deep-sequenced dataset of the same plasma samples suggests a wide range of ctDNA content (15% of samples with <0.5% ctDNA—AVENIO ctDNA Analysis Kit's limit of detection) so they are a sufficiently challenging set to evaluate this approach. A whole genome sequencing data analysis pipeline was developed that carries out the standard QC steps (fastq quality check, adapter trimming, duplicate removal) and computes relevant global metrics for downstream analysis.

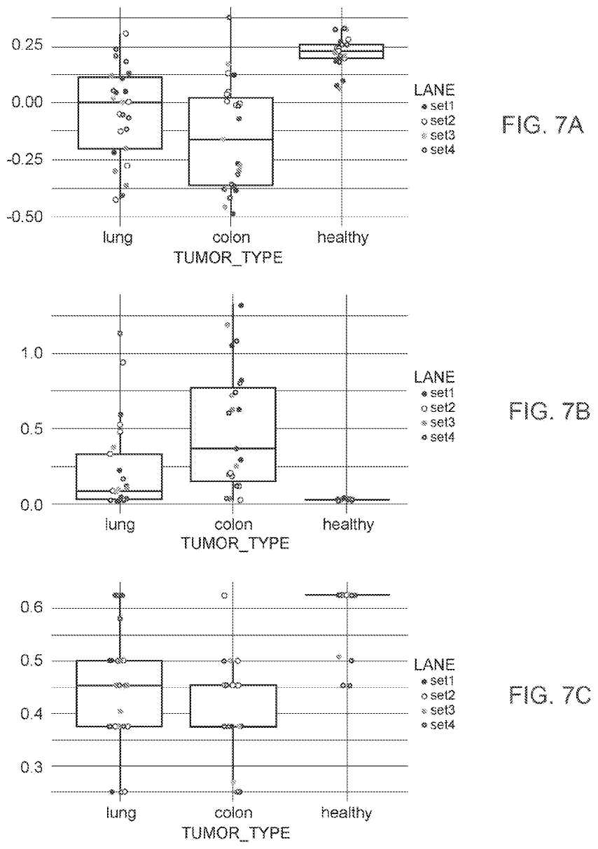

FIGS. 7A-7C show the power of a univariate analysis of global sequencing feature summaries to separate out the colon and lung cancer datasets from normal samples. For example, FIG. 7A shows a fragment score (PCA axis 1) between lung cancer, colon cancer, and normal data sets. FIG. 7B shows a copy number amplification score based on read depth analysis between lung cancer, colon cancer, and normal data sets. FIG. 7C shows an allelic imbalance score (e.g., the median p-value) between lung cancer, colon cancer, and normal data sets. As demonstrated, individually each of the global sequencing feature summaries can discrimination between cancer samples (samples comprising ctDNA) and normal samples (samples with no ctDNA). Other features and metrics discuss herein were also found to show various capabilities to discriminate between cancer samples and normal samples.

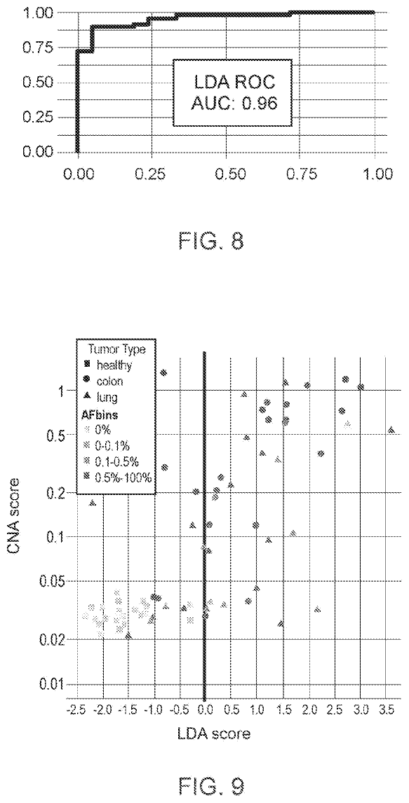

In a multivariate analysis, at least two features or metrics are combined into linear discriminant analysis classifier and demonstrate a capability to discriminate normal samples (samples with no ctDNA) from cancer samples (samples comprising ctDNA) with greater specificity and sensitivity. For example, a 3-fold cross validated linear discriminant analysis classifier was used to establish performance to discriminate between normal samples and cancer samples. FIG. 8 shows the Receiver Operator Characteristics (ROC) area under the curve (AUC) for this classifier, and it shows that >70% sensitivity (true positive rate) is achievable with 100% specificity (false positive rate=0). FIG. 9 shows the linear discriminant analysis score and copy number alteration score for each sample, colored by AF value of known somatic single-nucleotide variants (SNVs) in the sample. As shown, there are some samples that are correctly classified as cancer samples that have no detectable SNVs and there are several samples with SNV AFs <0.5% (below LOD) that are also reliably classified as cancer samples.

### VIII. Diagnostic Assay and Treatment

In various embodiments, techniques are provided for determining whether a subject has minimal residual disease based on the classification of the sample of cell free DNA as the first class or the second class by techniques disclosed herein. Some embodiments further encompass techniques for predicting a clinical outcome of a treatment regimen for the subject or providing a prognosis of cancer in the subject based on the determination of minimal residual disease. For example, once the sample is classified as the first class or the second class, the classification can be used to determine the presence of minimal residual disease in the subject.

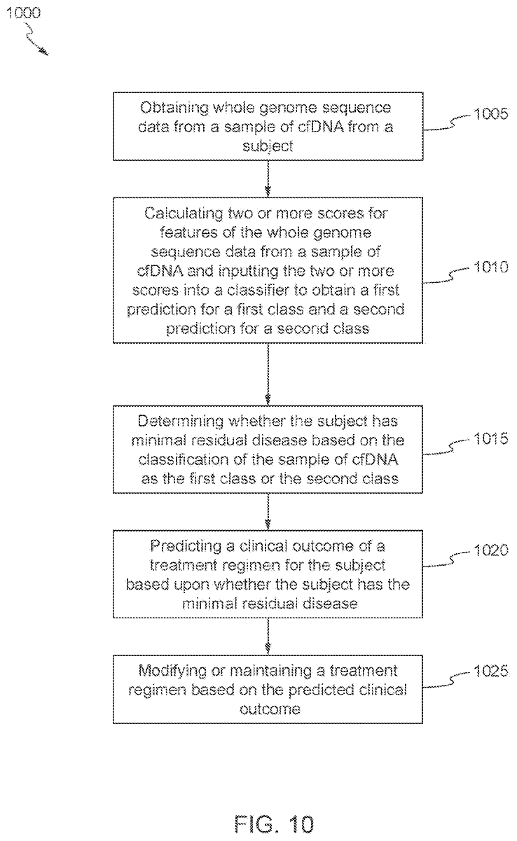

FIG. 10 shows a flowchart 1000 that illustrates processes and operations for diagnosing a patient with minimal residual disease. Individual embodiments may be described as a process which is depicted as a flowchart, a flow diagram, a data flow diagram, a structure diagram, or a block diagram, as previously described with respect to FIG. 1. The processes depicted in flowchart 1000 include some or all of the steps performed in flowchart 100 described with respect to FIG. 1 and may be implemented by the architecture, systems, and techniques depicted in FIGS. 2 and 3. At step 1005, whole genome sequence data is obtained from a sample of cfDNA from a subject (e.g., a patient). The whole genome sequence data includes a plurality of whole genome sequence reads. In some embodiments, the whole genome sequence data are obtained using a diagnostic assay. The assay can be created in a variety of ways and use various techniques, such as PCR, sequencing, hybridization arrays, and unique molecule identifiers. The assay should be able to detect ctDNA at pre-treatment levels. In some embodiments, the assay can be created as part of a kit that comprises reagents necessary for obtaining whole genome sequence data from the ctDNA. For example, the kit may comprise oligonucleotides such as probes and amplification primers specific for whole genome sequences. In some embodiments, the kit further comprises reagents necessary for the performance of amplification and detection assay, such as the components of PCR, a real-time PCR, or transcription mediated amplification (TMA). In some embodiments, the whole genome oligonucleotides are detectably labeled. In such embodiments, the kit comprises reagents for labeling and detecting the label. For example, if the oligonucleotides are labeled with biotin, the kit may comprise a streptavidin reagent with an enzyme and its chromogenic substrate.

At step 1010, two or more scores for features of the whole genome sequence data obtained from a sample of cell free DNA are calculated and input into a classifier to obtain a first prediction for a first class and a second prediction for a second class. In some embodiments, the features include the fragment size of the cfDNA, the relative read depth of the plurality of whole genome sequence reads, a germline allelic imbalance of the whole genome sequence reads, or a combination thereof. In other embodiments, the features include (i) a fragment size of the cfDNA, (ii) relative read depth of the plurality of whole genome sequence reads, (iii) germline allelic imbalance, (iv) softclipping rates, (v) rates of substitution types, (vi) overall predicted somatic mutation counts, (vii) rates of discordant reads, (vi) relative LINE/SINE element read depth, or combinations thereof. In some embodiments, the first class is the sample of cell free DNA includes circulating tumor DNA and the second class is the sample of cell free DNA does not include the circulating tumor DNA. At step 1015, whether the subject has minimal residual disease is determined based on the classification of the sample of cell free DNA as the first class or the second class. The minimal residual disease is the presence of residual tumor that remains in the subject during treatment or after treatment. For example, if the sample of cell free DNA is classified as the first class where the sample of cell free DNA includes circulating tumor DNA, then it may be determined that the subject has minimal residual disease. Alternatively, if the sample of cell free DNA is classified as the second class where the sample of cell free DNA does not include circulating tumor DNA, then it may be determined that the subject does not have minimal residual disease.

At step 1020, a clinical outcome of a treatment regimen for the subject is predicted based upon whether the subject has the minimal residual disease. Several studies have confirmed the importance of assessing the potential presence of minimal residual disease during or following a treatment regimen, to aid in predicting clinical outcomes of patients. For example, patients who do not exhibit sustained minimal residual disease, fare significantly better than patients who exhibit sustained minimal residual disease. At step 1025, upon determining the subject does have minimal residual disease and predicting a negative clinical outcome, the treatment regimen of the subject may be modified. Alternatively, upon determining the subject does not have minimal residual disease and predicting a positive clinical outcome, the treatment regimen of the subject may be maintained.

1. A method comprising:
(a) obtaining, by a data processing system, whole genome sequence data from a sample of cell free DNA from a subject, wherein the whole genome sequence data includes a plurality of whole genome sequence reads;
(b) calculate, by the data processing system, two or more metrics from at least a majority of the plurality of genome sequence reads, wherein a first metric of the two or more metrics is:
(i) a fragment size of the cell free DNA,
(ii) relative read depth of the plurality of whole genome sequence reads, or
(iii) germline allelic imbalance;
(c) inputting, by the data processing system, the two or more metrics into a classifier to obtain a first prediction for a first class and a second prediction for a second class, wherein the first class is the sample of cell free DNA includes circulating tumor DNA and the second class is the sample of cell free DNA does not include the circulating tumor DNA; and
(d) classifying, by the data processing system, the sample of cell free DNA as the first class or the second class based on the first prediction and the second prediction.

2. The method of claim 1, wherein a second metric of the two or more metrics is:
(i) a fragment size of the cell free DNA,
(ii) relative read depth of the plurality of whole genome sequence reads, or
(iii) germline allelic imbalance; and
wherein the second metric is different from the first metric.

3. The method of claim 1, wherein the classifier is a linear discriminant analysis.

4. The method of claim 1, wherein a third metric is calculated and input into the classifier, the third metric is:
(i) a fragment size of the cell free DNA,
(ii) relative read depth of the plurality of whole genome sequence reads, or
(iii) germline allelic imbalance; and
wherein each of the first metric, the second metric, and the third metric are different metrics.

5. The method of claim 1, wherein the fragment size of the cell free DNA is calculated by normalizing cell free DNA fragment sizes obtained in the sample thereby obtaining a probability density function value.

6. The method of claim 1, wherein the fragment size of the cell free DNA comprises a ratio of regions within a probability density function.

7. The method of claim 6, wherein the ratio of regions within the probability density function comprises a ratio of probability of cell free DNA fragment size of between about 116 and about 156 nucleotides in length and a ratio of probability of cell free DNA fragment size around a mode of between about 164 and about 168 nucleotides in length.

8. The method of claim 1, wherein the fragment size of the cell free DNA is a statistical fragment score calculated by:
(i) normalizing cell free DNA fragment sizes obtained in the sample thereby obtaining a probability density function value;
(ii) determining a log of values for the cell free DNA fragment sizes and first differences between consecutive cell free DNA fragment sizes;
(iii) removing at least 20 of lowest cell free DNA fragment sizes to obtain a remaining cell free DNA fragment sizes; and
(iv) determining a first principal component axis of the remaining cell free DNA fragment sizes as compared to cell free DNA that does include the circulating tumor DNA and a cell free DNA that does not include the circulating tumor DNA.

9. The method of claim 1, wherein the relative read depth of the plurality of whole genome sequence reads is calculated by:
(i) preprocessing of cell free DNA fragment size sequence read counts to obtain a set of normalized cell free DNA fragment size sequence read counts;
(ii) determining a median read depth per chromosome arm for the set of normalized cell free DNA fragment size sequence read counts; and
(iii) determining a maximum of the median read depth per chromosome arm to obtain a copy number amplification score.

10. The method of claim 9, wherein the preprocessing comprises:
(i) mapping sequence read counts from various samples into windows having predetermined sizes;
(ii) filtering sequence read counts in each window based on one or more factors to obtain a set of remaining cell free DNA fragment size sequence read counts for each window;
(iii) correcting for guanine-cytosine content and mappability biases in each window; and
(iv) normalizing remaining cell free DNA fragment size sequence read counts in each window against sequence data from cell free DNA samples that do include circulating tumor DNA.

11. The method of claim 1, wherein the germline allelic imbalance is calculated using a statistical model, which is preferably a binomial probability model to determine a median probability value for one or more germline allelic imbalance sites in the sample of cell free DNA, and to obtain an allelic imbalance score.

12. The method of claim 11, wherein if the median probability value for the one or more germline allelic imbalance sites is below a predetermined significance level, the median probability value is indicative of an allelic imbalance at the one or more germline sites in the sample of cell free DNA.

13. The method of claim 1, further comprising predicting, by the data processing system, whether the subject has minimal residual disease based on the classification of the sample of cell free DNA as the first class or the second class.

14. A method of diagnosing a patient with minimal residual disease comprising:
(a) calculating two or more scores for features of whole genome sequence data obtained from a sample of cell free DNA from a subject, wherein the features include: (i) a fragment size of the cell free DNA, (ii) relative read depth of the plurality of whole genome sequence reads, (iii) germline allelic imbalance, (iv) softclipping rates, (v) rates of substitution types, (vi) overall predicted somatic mutation counts, (vii) rates of discordant reads, (vi) relative LINE/SINE element read depth, or combinations thereof;
(b) inputting, by the data processing system, the two or more scores into a classifier to obtain a first prediction for a first class and a second prediction for a second class, wherein the first class is the sample of cell free DNA includes circulating tumor DNA and the second class is the sample of cell free DNA does not include the circulating tumor DNA;
(c) classifying, by the data processing system, the sample of cell free DNA as the first class or the second class based on the first prediction and the second prediction; and
(d) determining, by the data processing system, whether the subject has minimal residual disease based on the classification of the sample of cell free DNA as the first class or the second class.

15. A system comprising:
one or more processors; and
a memory accessible to the one or more processors, the memory storing a plurality of instructions executable by the one or more processors, the plurality of instructions comprising instructions that when executed by the one or more processors cause the one or more processors to perform a method of claim 1.

16. A computer product comprising a computer readable medium storing a plurality of instructions for controlling a computer system to perform a method of claim 1.
